(19) United States
(12) Patent Application Publication
(10) Pub. No.: US 2025/0276114 A1
(43) Pub. Date: Sep. 4, 2025

(54) In-field Emergency Blood Transfusion System and Method

(71) Applicant: Thomas M. LaMorte, Yardley, PA (US)

(72) Inventor: Thomas M. LaMorte, Yardley, PA (US)

(21) Appl. No.: 19/066,131

(22) Filed: Feb. 27, 2025

Related U.S. Application Data
(60) Provisional application No. 63/560,154, filed on Mar. 1, 2024.

(51) Int. Cl. A61M 1/02

(52) U.S. Cl. CPC A61M 1/024; A61M 2205/18; A61M 2230/06

(57) ABSTRACT

A system and method of transfusing blood from a donor source to a patient. To facilitate the transfusion, a transfer tube is provided. One end of the transfer tube is connected to the donor source and the opposite end is intravenously connected to the patient. A pump assembly is provided that contains a pump and a controller. The transfer tube is engaged with the pump assembly wherein the pump, when activated, acts upon the transfer tube to move blood. The controller monitors the blood volume moved and automatically stops the pump once a predetermined volume of blood has been transferred. If the donor source is a person, the predetermined volume is between 400 milliliters and 450 milliliters. The flow rate of the pump is preferably 100 milliliters per minute. This transfusion rate can be increased by synchronizing the pump to the heart rhythm of the patient.

### RELATED APPLICATION

This application claims the benefit of U.S. Provisional Patent Application No. 63/560,154, filed Mar. 1, 2024.

### BACKGROUND OF THE INVENTION

### 1. Field of the Invention

In general, the present invention relates to blood transfusion systems that are used in emergency situations, such as during natural disasters or on the battlefield. More particularly, the present invention relates to transfusion systems with pumps and tubes that can transfer blood from a donor source to a person in need in a highly expedited manner.

### 2. Prior Art Description

The average adult male human body holds about 2000 milliliters of blood. If a person loses one-fifth of their blood they are likely to go into hemorrhagic shock and lose consciousness. If a person loses one fourth of their blood, they are likely to die from exsanguination. Likewise, regardless of blood loss, if a person's blood pressure drops below 70/30 they are in danger of dying. In addition, even if the loss of blood does not cause death, the longer the state of hemorrhagic shock lasts, the more damage is caused to the body. Prolonged hemorrhagic shock can cause heart damage, brain damage, tissue loss and gangrene.

Blood loss to the point of exsanguination is typically caused from physical trauma, such as a gunshot wound, a shrapnel wound, or an impalement. The loss of blood is directly related to the location and size of the wound. In many instances, the wound can be bound to a degree where blood loss is manageable. However, if the patient has already lost too much blood, the blood must be replaced before the body passes into shock or passes from shock to death.

In cases of rapid blood loss, patients are typically treated using a blood transfusion. An intravenous line is inserted into the patient and a stored bag of blood or plasma is attached to the line. The blood or plasma then flows into the patient using the force of gravity. The typical flow of blood into the body using this system is typically around 70 ml per minute. This rate can be increased by using an intravenous line pump. However, even with such a pump, the maximum flow rate is around 80 ml per hour. Most intravenous line pumps are designed to introduce intravenous fluids, such as medicated saline solutions into a patient. Intravenous line pumps are designed to supply controlled volumes that are often measured in drops per minute, which translates to milliliters per hour. Intravenous line pumps are therefore designed for small flow rates and are not intended to maximize flow from a fluid source to the patient. Such prior art intravenous line pumps are exemplified by U.S. Pat. No. 9,677,555 to Kamen et al., U.S. Pat. No. 10,342,921 to Barnes et al., U.S. Pat. No. 5,399,166 to Laing and U.S. Patent Application Publication No. 2004/0064097 to Peterson.

Although such prior art intravenous line pumps can be considered transportable, they are still intended for use in a hospital or other stable environment, such as a field hospital, where there are clean conditions and access to external power. Such prior art transfusion pumps are also designed for use on stabilized patents where the flow rate through the intravenous line can be kept under 300 ml per hour. Such prior art pump systems tend to contain drip chambers that only work when vertically oriented and therefore are poorly suited for battlefield use. In addition, such prior art systems cannot operate in battlefield conditions where there is no available external power, and the unit may be soaked with rain, snow, seawater, blood, and battlefield debris.

In many circumstances, a patient may need blood immediately and no stored blood or plasma is available. This is often the case on the battlefield. The transfusion is often conducted on an active battlefield while cramped behind limited cover. In such a circumstance, a medic may have to resort to a direct donor-to-patient blood transfusion, which is often referred to as an in-field blood transfusion. In an in-field blood transfusion, blood is directly transferred from a donor to a patient. The donors must remain available and stationary for the period of transfusion, which can be as long as a half hour per donor. This is highly impractical and often impossible for soldiers on a battlefield.

A need therefore exists for an improved system and methodology of performing an in-field blood transfusion, wherein the time required to transfer blood from a donor to a patient can be greatly reduced. A need also exists for an improved system and methodology of performing an in-field blood transfusion that can operate in any orientation and robust enough to function without external power in a very wet and contaminated environment. This need is met by the present invention as described below.

### SUMMARY OF THE INVENTION

The present invention is a system and method of rapidly transfusing blood directly from a donor source to a patient. The donor source can be a living person or a prefilled bag of blood or blood plasma. To facilitate the transfusion, a transfer tube is provided. One end of the transfer tube is connected to the donor source. The opposite end of the transfer tube is intravenously connected to the patient.

A pump assembly is provided that contains a pump, at least one battery for powering the pump, and a controller for controlling the pump. The pump, battery and controller are encased in a common waterproof housing. The transfer tube is engaged with the pump assembly wherein the pump, when activated, acts upon the transfer tube to move blood from the donor source to the patient.

The controller monitors blood volume moved by the pump and automatically stops the pump once a predetermined volume of blood has been transferred. If the donor source is a person, the predetermined volume is between 400 milliliters and 450 milliliters. The flow rate of the pump is preferably 100 milliliters per minute. As such, a transfusion from a human donor to a patient should take no longer than 4.5 minutes. This transfusion rate can be increased by synchronizing the pump to the heart rhythm of the patient. The rapid rate of transfusion represents a significant advancement in the art that can save many patients from passing into hemorrhagic shock or passing from hemorrhagic shock to death.

### BRIEF DESCRIPTION OF THE DRAWINGS

For a better understanding of the present invention, reference is made to the following description of exemplary embodiments thereof, considered in conjunction with the accompanying drawings, in which:

FIG. 1 shows an image of two soldiers using an exemplary embodiment of the present invention blood transfusion system in the field;

FIG. 2 shows an overview of an exemplary embodiment of the present invention blood transfusion system shown in conjunction with a donor and a patient;

FIG. 3 is a schematic of the major components contained within the pump assembly of the present invention;

FIG. 4 us a block diagram schematic showing the primary operational components contained within the pump assembly;

FIG. 5 is a graph that shows the synchronization of the pump to the diastolic cycle of the patient's heartbeat;

FIG. 6 is a block diagram that outlines the methodology of using the present invention blood transfusion system;

FIG. 7 is a block diagram that outlines the operational methodology of the blood transfusion system; and

FIG. 8 shows an overview of an alternate exemplary embodiment of the present invention blood transfusion system.

### DETAILED DESCRIPTION OF THE DRAWINGS

Although the present invention system and methodology can be embodied in many ways, only a few exemplary embodiments are illustrated. The exemplary embodiments are being shown for the purposes of explanation and description. The exemplary embodiments are selected in order to set forth some of the best modes contemplated for the invention. The illustrated embodiments, however, are merely exemplary and should not be considered limitations when interpreting the scope of the appended claims.

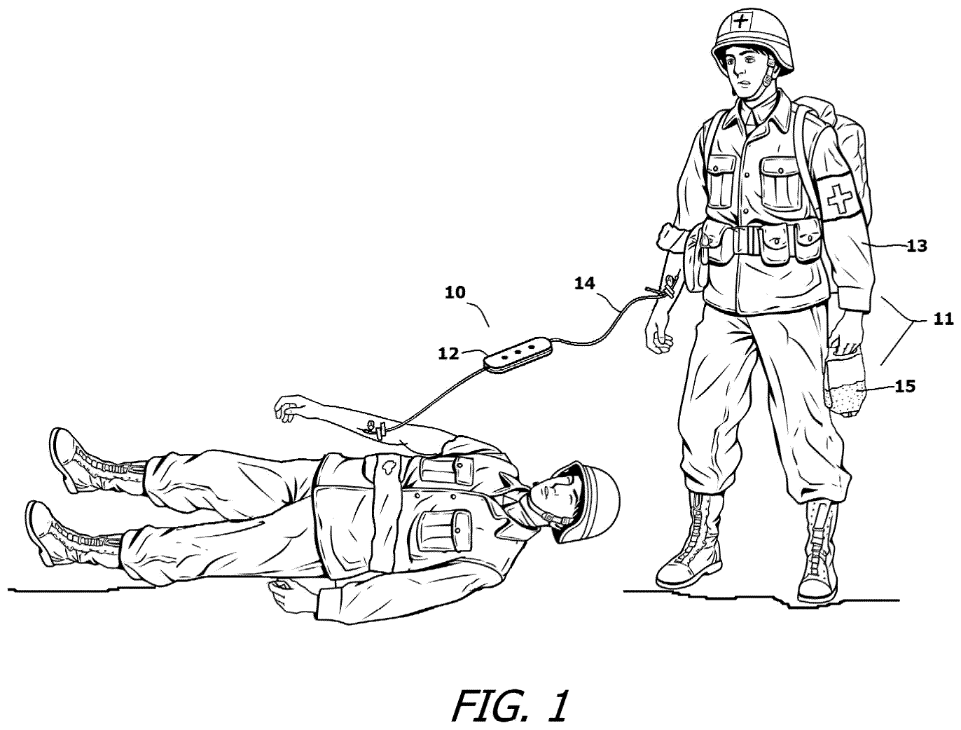

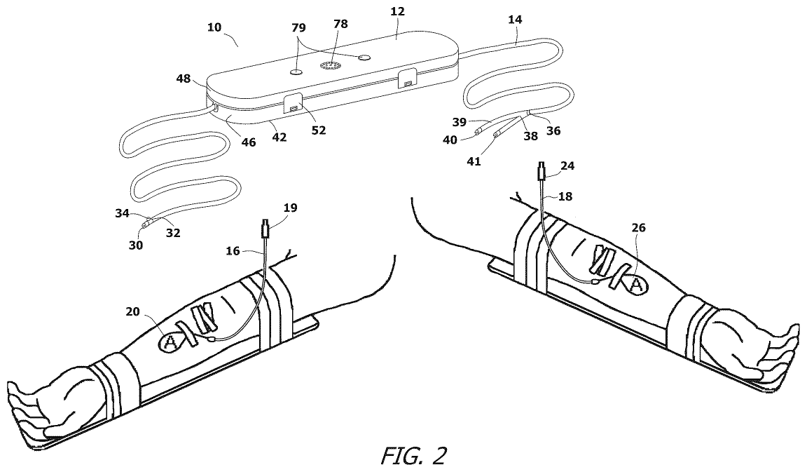

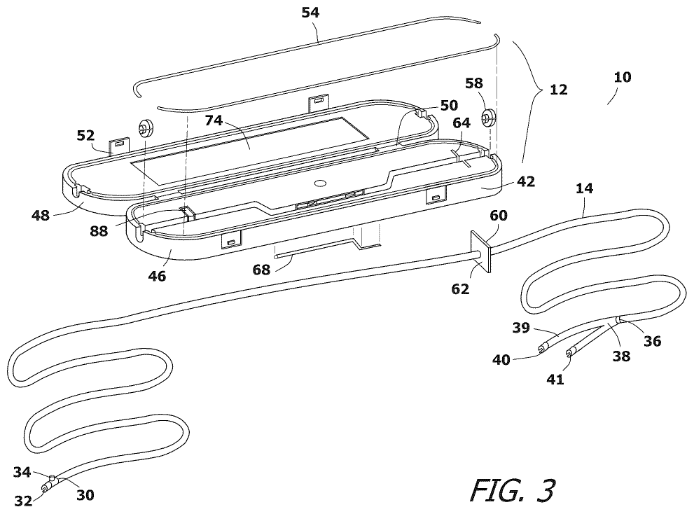

Referring to FIG. 1, FIG. 2, and FIG. 3, a first embodiment of an emergency blood transfusion system 10 is shown. The emergency blood transfusion system 10 contains a portable pump assembly 12, a transfer tube 14, and two intravenous ports 16, 18. These elements are transferred together in a unit package that is carried by a field medic or other medically trained personnel. The purpose of the emergency blood transfusion system 10 is to transfer a monitored volume of blood directly from a donor source to a patient in the shortest amount of time possible. The donor source 11 can be a person 13 or a prefilled bag 15 of blood or plasma.

To utilize the emergency blood transfusion system 10, the first intravenous port 16 is inserted into a patient who is in need of blood. The first intravenous port 16 is firmly taped in place to prevent any inadvertent disengagement caused by the pressures generated by the emergency blood transfer system 10. The first intravenous port 16 is a standard IV port that has a first tube connector 19. A patient blood-type identifying graphic 20 is provided on the first intravenous port 16 for a purpose later explained. The blood-type identifying graphic 20 contains the letter(s) and polarity of the blood type along with a color scheme associated with that blood type. For example, many organizations including the American Red Cross® use pink to color code A+ blood and light blue to color code O-blood.

Blood or plasma is drawn from the donor source 11. The donor source 11 can be a person 13 or a prefilled bag 15 of blood or plasma, as indicated in FIG. 1. If no preexisting bag of blood or plasma is available, the second intravenous port 18 is connected to a person 13. If multiple donors are available, each can be fitted with such an intravenous port. Each second intravenous port 18 preferably consists of a short tube segment 22 that terminates with a second tube connector 24. A donor blood-type identifying graphic 26 is provided for a purpose later explained. The identifying graphic 26 contains the letter(s) and polarity of the donor's blood type along with a color scheme associated with that blood type.

A transfer tube 14 is provided. The transfer tube 14 is a medical grade silicone tube or PTFE tube with a preferred inner diameter of between 1.6 mm and 5.0 mm. The transfer tube 14 is capable of interacting with the portable pump assembly 12 so that the portable pump assembly 12 can move fluid through the transfer tube 14. The transfer tube 14 can have any length between one and three meters. The transfer tube 14 has a first end 30 that terminates with a first port connector 32. The first port connector 32 is configured to selectively interconnect to the first tube connector 19 on the patient's first intravenous port 16. The first port connector 32 also has a venting valve 34 that enables the transfer tube 14 to be selectively vented. This enables air to exit the transfer tube 14 at the venting valve 34 during a priming procedure.

The opposite second end 36 of the transfer tube 14 preferably has a Y-junction 38. Each arm 39, 41 of the Y-junction terminates with a second port connector 40 that can be attached to the second tube connector 24 on the intravenous port 18 of the donor. The Y-junction 38 enables the transfer tube 14 to be connected both to a first donor and/or to a bag of blood or plasma.

Alternatively, the Y-junction 38 enables the transfer tube 14 to be connected to a first donor and then to a second donor just prior to the disconnection of the first donor. This enables a constant flow of blood from subsequent donors without having to pause the flow of blood and without needing to reprime the transfer tube 14. It also allows a bag of blood or plasma to be substituted for a donor and vice versa.

The flow of blood through the transfer tube 14 is not controlled by gravity and is unaffected by gravity or orientation. Rather, the portable pump assembly 12 is used. As is shown best in FIG. 2 and FIG. 3, the portable pump assembly 12 has a containment housing 42. The containment housing 42 is designed for battlefield use and provides a hermetic barrier around the electronics and other working components in accordance with the casing standard outlined in military standard specification MIL C-4150J. The containment housing 42 is also crushproof by military standards. That is, the containment housing 42 is capable or maintaining integrity under 100 psi of pressure. Thus, the containment housing 42 can be stepped on or even run over by a vehicle without damage.

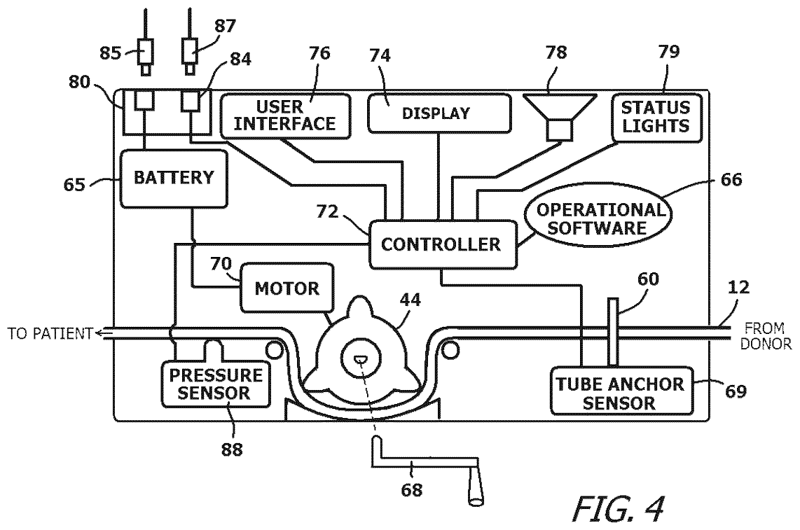

Referring to FIG. 2 and FIG. 3 in conjunction with FIG. 4, it will be understood that the containment housing 42 is used to hold an electromechanical pump 44, as well as the power source, electronics, and user interface for operating the electromechanical pump 44. In the shown embodiment, the containment housing 42 is shown having a clamshell design wherein a first housing section 46 closes over a second housing section 48 using a hinged connection 50. The clamshell design minimizes the overall size of the portable pump assembly 12. In a closed configuration, the first housing section 46 can be locked to the second housing section 48 using traditional latches 52. An O-ring seal 54 is provided between the first housing section 46 and the second housing section 48. The O-ring seal 54 prevents moisture and/or debris from entering the containment housing 42 when the containment housing 42 is locked into its closed configuration.

A groove 56 is formed in the first housing section 46. When the containment housing 42 is in an open configuration, the full length of the groove 56 is readily accessible. Plug elements 58 may be provided at opposite ends of the groove 56. The plug elements 58 block the groove 56 and prevent moisture and debris from entering the groove 56 when the containment housing 42 is in its closed configuration. When the containment housing 42 is in its open configuration, the plug elements 58 can be manually removed to fully open the groove 56. Secondary seals can be provided that form a seal around the transfer tube 14 once the transfer tube 14 is set into the groove 56.

The groove 56 has a width that is slightly larger than the diameter of the transfer tube 14. Accordingly, the groove 56 is sized to receive a section of the transfer tube 14 and hold the transfer tube 14 in position where it can be acted upon by the electromechanical pump 44. A tube anchor 60 is used to hold the transfer tube 14 in a fixed position relative to the electromechanical pump 44. The tube anchor 60 is a flange 62 that is affixed to the transfer tube 14 at a specific point. A flange slot 64 is formed in the containment housing 42 along the groove 56 that receives and retains the tube anchor 60. The engagement of the tube anchor 60 into the flange slot 64 prevents the transfer tube 14 from moving relative to the containment housing 42 as the electromechanical pump 44 applies forces to the transfer tube 14. A sensor 69 is present in the flange slot 64 that can detect the presence of the flange slot 64 and provide a signal.

In FIG. 4, the electromechanical pump 44 is shown as a peristaltic pump. Such a pump is preferred. However, other pumps, such as lobe pumps or gear pumps can also be used provided they are modified to move fluid within a flexible tube without directly contacting the fluid in the tube. The electromechanical pump 44 is used to move blood and/or plasma through the transfer tube 14. The electromechanical pump 44 can engage the transfer tube 14 anywhere that the tube anchor 60 is provided. The electromechanical pump 44 is primarily powered by a battery 65 that is within the containment housing 42. The battery 65 is preferably a rechargeable battery, such as the ABC batteries utilized by the U.S. Military. The rate that the electromechanical pump 44 moves blood/plasma through the transfer tube 14 is controlled by operational software 66. The operational software 66 can also monitor the operational rate of the electromechanical pump 44 over time to calculate the volume of blood/plasma pumped during that time. The volume of blood/plasma pumped is stored by the operational software 66 for consideration by medical personnel attending to the patient. It is understood that during emergency use, the batteries 65 of the portable pump assembly 44 may drain rapidly from constant use. Replacement batteries or the ability to recharge the batteries may not exist. As such, the electromechanical pump 44 preferably has the ability to receive a manual crank 68 so that the electromechanical pump 44 can be manually turned when required.

The portable pump assembly 12 is selectively engaged with the transfer tube 14. The electromechanical pump 44 has a motor 70 that normally powers the electromechanical pump 44. The motor 70 is connected to a controller 72. The controller 72 operates the motor 70 and uses the operational software 66 to monitor the rate at which the electromechanical pump 44 is turning and the number of times that the peristaltic pump turns. As such, the controller 72 can calculate the flow rate of blood/plasma through the transfer tube 14 and/or the volume of blood/plasma that has been transferred. These values can be viewed on a display 74 having a user interface 76. The display 74 is capable of visually displaying data, instructions, and warnings. In addition to the display 74, a speaker 78 and status light 79 are provided. The speaker 78 can audibly broadcast data, instructions, and alarms. The status lights 79 preferably contain a green light and a red light to quickly display the operational status of the system 10.

The pressure in the transfer tube 14 between the electromechanial pump 44 and the patient is monitored by the controller 72. A pressure sensor 88 is provided that presses against the transfer tube 14 within the groove 56. The pressure sensor 88 is biased against the transfer tube 14. As the pressure in the transfer tube 14 changes, the resistance to the bias changes and the pressure within the transfer tube 14 can be determined without physically contacting the blood or plasma flowing through the transfer tube 14.

Optionally, the portable pump assembly 12 can have an input bay 80 that is protected by a waterproof closure 82. In the input bay 80 are input ports 84, 85 that lead to the controller 72 and/or battery 65. The input port 85 for the battery 65 is a power lead that can engage a power cable to recharge the battery 65 or provide power in place of the battery 65. A least one data input 84 is provided that leads to the controller 72. The data input 84 is used to connect the controller 72 to an outside computer for program updates, data downloads, and diagnostic interrogations. The data input 84 can also be used to connect the controller 72 to an auxiliary patient sensor 86, such as an auxiliary patient sensor 87 that is connected to the patient in need of blood. If the controller 72 can read pulse data from the auxiliary patient sensor 87, then the operational software 66 can determine the pulse rate of the patient. The controller 72 can also use the auxiliary patient sensor 87 to detect if a pulse stops and sound an alarm.

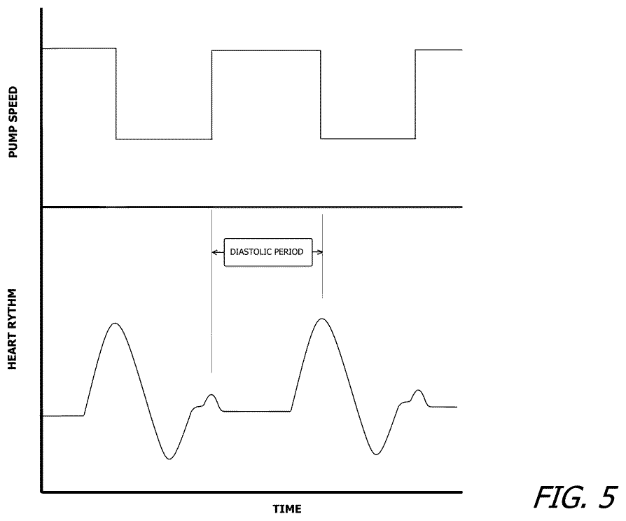

Referring to FIG. 5 in conjunction with FIG. 4, it will be understood that if the controller 72 is connected to an auxiliary patient sensor 87, the operational software 66 can determine the timing of the systolic and diastolic cycle of the heart's pumping rhythm. During the systolic cycle, the heart contracts and blood pressure momentarily increases to its maximum. During the diastolic cycle, the heart expands and fills with blood, therein causing the blood pressure to momentarily decrease to its minimum. The operational software 66 operates the electromechanical pump 44 to be in rhythm with the patient's heart. The controller 72 can pulse the electromechanical pump 44 so that it pumps blood/plasma more rapidly during the diastolic cycle of the heart and less rapidly during the systolic cycle of the heart. By synchronizing the electromechanical pump 44 to the natural rhythm of the heart, the rate of blood/plasma flow into the patient can be increased by over 25 percent as compared to a constant flow pump.

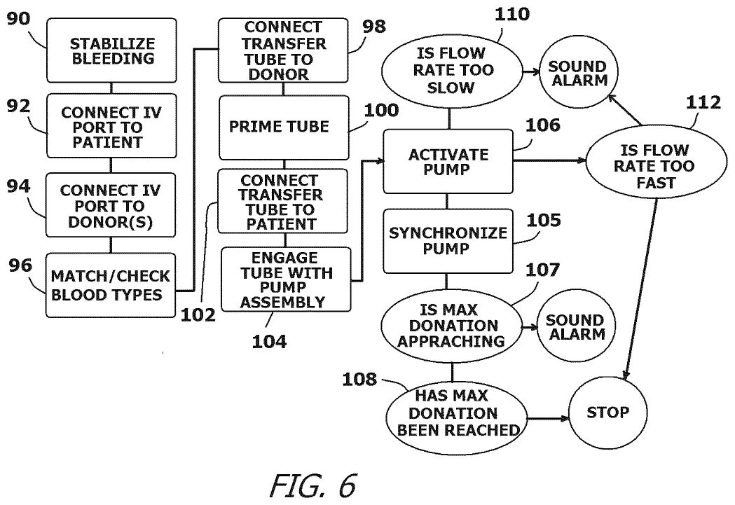

Referring to FIG. 6, in conjunction with FIG. 2 and FIG. 3, it can be seen that to utilize the present invention transfusion system 10, a patient's bleeding rate is stabilized and a first intravenous port 16 is connected to the patient. See Block 90 and Block 92. A separate second intravenous port 18 is attached to a donor. See Block 94. The first intravenous port 16 and the second intravenous port 18 contain identifying graphics 20, 26. The identifying graphics 20, 26 can be color codes, number codes or other indicators of the person's blood type. In this manner, if there are many patients and many donors, a medic in an emergency situation can match the identifying graphics 20, 26 and quickly identify what donors are proper to donate to a specific patient. See Block 96.

Once a donor is matched to a patient, a transfer tube 14 can be connected to the donor. The transfer tube 14 is vented to allow the transfer tube 14 to fill with blood from the donor. Once primed with blood, the transfer tube 14 is connected to the first intravenous port 16 of the patient. See Block 98, Block 100, and Block 102. The transfer tube 14 is then engaged with the portable pump assembly 12. See Block 104. The portable pump assembly 12 is then activated. See Block 106. Once activated, the portable pump assembly 12 can draw blood from a donor at any rate that can be sustained by the donor. The preferred operating rate for an average adult male donor is 100 ml/min. As has been mentioned, the flow rate can be optionally varied so that the flow rate produced by the electromechanical pump 44 is synchronized with the pumping rhythm of the heart of the recipient. See Block 105.

The controller 72 in the portable pump assembly 12 monitors the flow of blood and automatically stops the flow once 450 milliliters of blood have been pumped. See Block 107 and Block 108. At the rate of 100 ml/min, the desired donation of 450 milliliters can be collected in only 4.5 minutes. If the flow of blood is pulsed in synchronization with the patient's heartbeat, the 450 milliliter of blood/plasma can be transferred in as little as 4 minutes. This is at least three times faster than a traditional gravity-fed blood transfusion from a blood bag. This increase in blood transfer efficiency greatly reduces the chances that a person who has lost blood will enter hemorrhagic shock or die of exsanguination. Since the volume of blood is monitored and controlled, the donor will not be drained of a dangerous amount of blood. The controller 72 will also monitor the flow rate and can sound alarms if the flow rate is too fast, indicating a disconnection or untreated hemorrhage, or too slow indicating a blockage. See Block 110 and Block 112.

As a donor approaches his/her maximum donation volume, the controller 72 can display and/or sound a warning. This enables a medic to connect a different donor to the transfer tube 14 so that the initial donor can be safely disconnected. By connecting a second donor or bag to the transfer tube 14 in this manner, no air gets introduced into the transfer tube 14 and no time is wasted repriming the transfer tube 14.

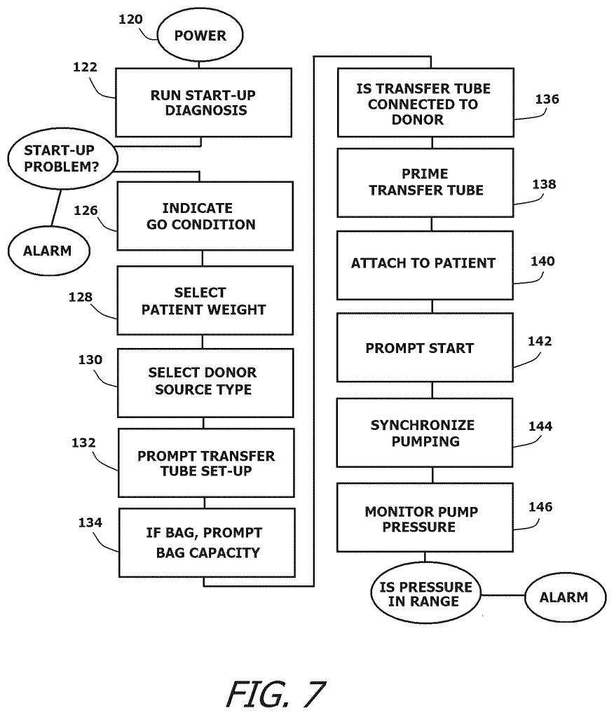

The controller 72 runs the operational software 66. Referring to FIG. 7 in conjunction with FIG. 4 and FIG. 3, the inputs of an exemplary version of the operational software 66 are shown. As is indicated by Block 120, the emergency blood transfusion system 10 is powered on. Upon power up, the operation software 66 runs a rapid diagnosis that checks the status of the system and the power available in the batteries 65. See Block 122 and Block 124. If the system diagnosis is successful and there is power, a green light is displayed on the display. See Block 126. This may also be accompanied with some sort of an audible signal.

The medical professional using the emergency blood transfusion system 10 is then prompted with a patient input prompt. In this prompt, the approximate weight of the patient is entered. See Block 128. This can be done using a menu selection presented on the display 74. For example, the prompt may indicate that the patient is 1. 100lbs-140 lbs, 2. 141 lbs-180 lbs, 3. 181 lbs-220 lbs 4. Over 220 lbs. The size of a patient is proportional to the volume of blood in that patent and can be used to determine a maximum transfusion volume.

The next prompt is a prompt to indicate if the donor source will be a live donor or a prefilled bag. See Block 130. Once the donor source is selected, the user is prompted to connect the transfer tube 14 to a donor source and then insert the transfer tube 14 into the groove 56 on the housing 42. See Block 132. If the donor source is a person, the operational software 60 sets a limit of 450 milliliters of blood to be drawn. If the donor source is a bag, a second prompt is created that asks for the volume of the bag. See Block 134.

Once the transfer tube 14 is set, the switch 69 in the flange slot 64 is triggered by the tube anchor 60. This informs the controller 72 that the transfer tube 14 is properly positioned in the containment housing 42. Once the transfer tube 14 is set, the operational software 66 generates a prompt asking if the transfer tube is connected to the donor source. See Block 136. If the prompt is answered in the affirmative, the operational software 66 prompts the user to open the venting valve 34 and the electromechanical pump 44 runs for a few seconds to prime the transfer tube 14. See Block 138. Once the transfer tube 14 is primed, the venting valve 34 is closed. An over pressure is soon created in the transfer tube 14 that is detected by the pressure sensor 88 and the controller 72 stops the electromechanical pump 44.

The controller 72 generates a prompt to attach the transfer tube 14 to the patient in need of blood. See Block 140. Once the transfer tube 14 is connected a “start” prompt is answered on the display and the electromechanical pump 44 starts pumping. See Block 142. If the controller 72 receives biofeedback from the patient, the pumping is synchronized to the heartbeat. See Block 144. This is a dynamically updated process since the heart may start beating faster or slower as blood supply in the patient increases. The pressure in the transfer tune is monitored during pumping. See Block 146 in FIG. 7 and Blocks 110 and 112 in FIG. 6.

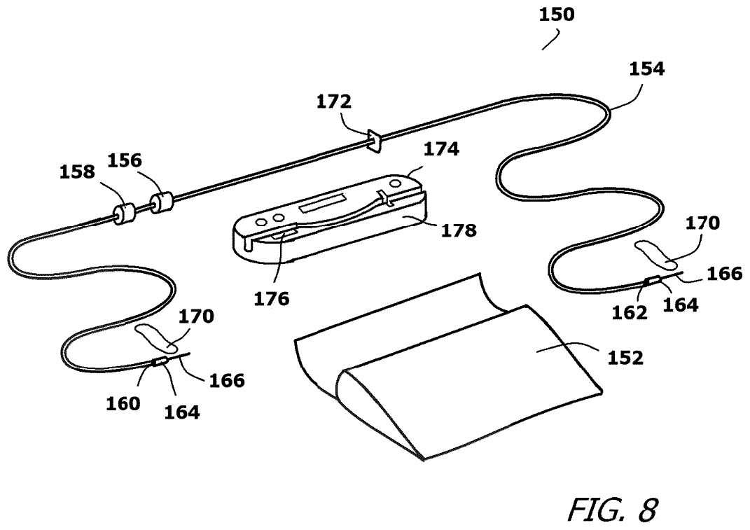

Referring now to FIG. 8, an alternate embodiment of the blood transfusion system 150 is shown. This blood transfusion system 150 is simplified and it can be carried in a sealed package 152 by a medic in the field. The blood transfusion system 150 includes a transfer tube 154. An in-line filter 156 and a one way valve 158 are provided on the transfer tube 154. The filter 156 and one way valve 158 prevents any clots or coagulations from passing into the patient and prevents any backflow into the donor.

The transfer tube 154 has a first end 160 and an opposite second end 162. Both ends 160, 162 terminate with connectors 164 that directly receive needle heads 166. In this manner, the patient and the donor need not be prepared with IV ports. Rather, the transfer tube 154 can be directly connected to a vein of the patient and an artery of the donor. Strips of tape 170 can be provided to hold the needle heads 166 in place and t50 identify blood type.

The transfer tube 154 also has a tube anchor 172 formed along its length. The transfer tube 154 is engaged with a portable pump assembly 174. The portable pump assembly 174 has a simplified design where the groove 176 for the transfer tube 154 is accessible on the top of the housing 178. Thus, no opening and closing of the housing 178 is required. Otherwise, the portable pump assembly 150 operates in the same manner as has been previously described.

It will be understood that the embodiments of the present invention that are illustrated and described are merely exemplary and that a person skilled in the art can make many variations to those embodiments. All such embodiments are intended to be included within the scope of the present invention as defined by the below claims.

What is claimed is:

1. A method of rapidly transfusing blood directly from a donor to a patient, said method comprising:
providing a transfer tube;
intravenously connecting said transfer tube to the donor;
intravenously connecting said transfer tube to the patient;
providing a pump assembly that contains a pump, at least one battery for powering said pump, and a controller for controlling said pump, wherein said pump, said controller and said at least one battery are encased in a common housing;
engaging said transfer tube with said pump assembly wherein said pump, when activated, acts upon said transfer tube to move blood from the donor to the patient,
wherein said controller monitors blood volume moved by said pump and automatically stops said pump once a predetermined volume of blood has been transferred.

2. The method according to claim 1, wherein said predetermined volume is between 400 milliliters and 450 milliliters

3. The method according to claim 1, wherein said transfer tube has a first end, a second end and an uninterrupted length between said first end and said second end.

4. The method according to claim 3, further including attaching an intravenous port to the patient, wherein said first end of said transfer tube terminates with a connector and wherein intravenously connecting said transfer tube to the patient includes connecting the connector to the intravenous port.

5. The method according to claim 3, further including attaching an intravenous port to the donor, wherein said second end of said transfer tube terminates with a connector and wherein intravenously connecting said transfer tube to the donor includes connecting the connector to the intravenous port.

6. The method according to claim 3, wherein said first end of said transfer tube terminates with a first connector and wherein intravenously connecting said transfer tube to the patient includes connecting the first connector to a first needle head, wherein said first needle head is used to produce a first intravenous blood connection with the patient.

7. The method according to claim 6, wherein said second end of said transfer tube terminates with a second connector and wherein intravenously connecting said transfer tube to the donor includes connecting the second connector to a second needle head wherein said second needle head is used to produce a second intravenous blood connection with the donor.

8. The method according to claim 3, wherein said transfer tube includes a vent valve proximate said first end and said method includes venting air from said transfer tube to remove air from the transfer tube after said transfer tube is intravenously connected to the donor.

9. The method according to claim 3, wherein said controller monitors pressure in said transfer tube between said pump and said patient, and said controller sounds an alarm should said blood pressure fall outside a preselected range.

10. The method according to claim 1, wherein said transfer tube has an anchor flange extending therefrom at a point between said first end and said second end, wherein said common housing includes a slot for receiving said anchor flange and a sensor that detects said anchor flange in said slot.

11. The method according to claim 1, further including detecting a heart rhythm of the patient and utilizing said heart rhythm to control said pump.

12. The method according to claim 1, further including providing a manual crank that is detachable from said common housing and utilizing said manual crank to power said pump should said at least one battery run out of power.

13. A method of rapidly transfusing fluid directly from a donor source to a patient, said method comprising:
providing a transfer tube having a first end, a second end, and an uninterrupted length between said first end and said second end;
connecting said transfer tube to the donor source;
intravenously connecting said transfer tube to the patient;
providing a pump assembly that contains a pump, at least one battery for powering said pump, and a controller for controlling said pump, wherein said pump, said controller and said at least one battery are encased in a portable waterproof housing;
engaging said transfer tube with said pump assembly wherein said pump, when activated, acts upon said transfer tube to move blood from the donor source to the patient,
wherein said controller monitors blood volume moved by said pump and automatically stops said pump once a predetermined volume of blood has been transferred.

14. The method according to claim 13, wherein said predetermined volume is selectively programmed into said controller.

15. The method according to claim 13, wherein said donor source is selected from a group comprising human donors, prefilled bags of blood, and prefilled bags of blood plasma

16. The method according to claim 13, wherein said transfer tube includes a vent valve proximate said first end and said method includes venting air from said transfer tube to remove air from said transfer tube after said transfer tube is intravenously connected to the donor.

17. The method according to claim 13, wherein said controller monitors pressure in said transfer tube between said pump and said patient, and said controller sounds an alarm should said pressure fall outside a preselected range.

18. The method according to claim 13, wherein said transfer tube has an anchor flange extending therefrom at a point between said first end and said second end, wherein said housing includes a slot for receiving said anchor flange and a sensor that detects said anchor flange in said slot.

19. The method according to claim 13, further including detecting a heart rhythm of the patient and utilizing said heart rhythm to control said pump.
